(19) United States
(12) Patent Application Publication
(10) Pub. No.: US 2025/0195663 A1
(43) Pub. Date: Jun. 19, 2025

(54) PROCESS FOR PRODUCING POLYMER-STABILIZED ANTIOXIDANT CRYSTALS

(71) Applicant: The Board of Trustees of the University of Illinois, Urbana, IL (US)

(72) Inventors: Hyunjoon KONG, Champaign, IL (US); Jonghwi LEE, Seoul (KR); Youngjun KIM, Saarbrucken (DE); Hee Sun HAN, Champaign, IL (US); Ryan Cree MILLER, Champaign, IL (US)

(21) Appl. No.: 18/980,734

(22) Filed: Dec. 13, 2024

Related U.S. Application Data
(60) Provisional application No. 63/610,510, filed on Dec. 15, 2023.

(51) Int. Cl. A61K 47/18; A61K 9/00; A61K 9/08; A61K 35/28; A61K 47/22; A61K 47/36

(52) U.S. Cl. CPC A61K 47/183; A61K 9/0019; A61K 9/08; A61K 35/28; A61K 47/22; A61K 47/36

(57) ABSTRACT

In one embodiment, the invention relates to a process for producing a crystalline antioxidant comprising: (1) providing an aqueous solution comprising a hydrophilic antioxidant; and a polymer selected from hyaluronate-dopamine, alginate-dopamine, chitosan-dopamine, poly(ethylene glycol) dopamine and their derivatives, or mixtures thereof; (2) forming a liquid droplet of the aqueous solution, the droplet having a diameter, wherein polymer-induced nucleation of the antioxidant occurs in the liquid droplets; (3) crystallizing the crystalline antioxidant by performing at least one of: (a) subjecting the liquid droplet to thermal cycling between a lower temperature and a higher temperature, wherein antioxidant crystal growth occurs at the lower temperature and crystal dissolution occurs at the upper temperature; and maintaining the thermal cycling until the droplet contains one or two antioxidant crystals, and (b) subjecting the liquid droplet to shear deformation using a shear-induced mixing-on-a-chip device, wherein a probability of nucleation and crystallization of each droplet is increased.

### GOVERNMENT LICENSE RIGHTS

This invention was made with government support under Grant number 1735252 and 1932192 awarded by the National Science Foundation. The government has certain rights in the invention.

### FIELD OF THE INVENTION

The invention relates to a process for producing crystalline antioxidants. More particularly, the invention relates to a process for producing polymer-stabilized hydrophilic antioxidants.

### BACKGROUND OF THE INVENTION

Mesenchymal stem cells (MSCs) have long been studied as potentially providing new generations of medicine due to their secretion of various therapeutic proteins and extracellular vesicles; however, the in vitro expansion required to attain enough cells for clinical applications often leads to cell heterogeneity, which is undesirable. In turn, changes in the cellular secretome and loss of therapeutic efficacy are observed. The heterogeneity in MSCs is further exacerbated by cellular senescence, which is heavily influenced by the oxidative state of the cell. Senescent cells stop multiplying but don't die off, instead remaining and continuing to release chemicals that can trigger inflammation. Moreover, senescent MSCs produce reactive oxygen species (ROS) and spread senescence to neighboring cells, compounding adverse effects on the secretome bio-efficacy. Many high throughput label-free approaches, such as density gradient centrifugation and microfluidic mechanical or hydrodynamic filtration, exist to separate senescent cells from primary MSC samples prior to expansion including. Nevertheless, these size-dependent techniques miss quiescent cells that can transition into senescent cells without proper intervention.

It is known that hydrophilic antioxidants, such as N-acetylcysteine (NAC) and ascorbic acid, can be added to cell culture media to reverse quiescence and prevent senescence spreading through reactive oxygen species (ROS) scavenging. However, most hydrophilic antioxidants are metabolized within 6 hours in media, leaving <5% bioactive. The increased and more frequent dosing for compensation leads to antioxidant stress, DNA damage in proliferating MSCs, and large efficacy variation. In addition, even micromolar fluctuations in the ROS levels in MSCs can lead to substantial changes in intracellular transcription and translation of therapeutic paracrine factors, driving the need for prolonged and sustained antioxidant release. Thus, there is an ongoing need for processes that crystallize hydrophilic antioxidants which avoid or minimize the above undesirable affects.

### SUMMARY OF THE INVENTION

It has unexpectedly been discovered that a synthesis process where hydrophilic antioxidants are stabilized with polymers, and the antioxidants are then crystallized from droplets which undergo a cyclic thermal treatment, produce crystalline antioxidants with excellent properties. Specifically, these crystalline materials, known as a microcrystal assembly for senescence control (MASC), demonstrate significantly extended and uniform release profiles, and minimize ROS-triggered senescence, as confirmed by senescent biomarkers, exosome secretion, and paracrine factor-encoding gene expression.

In one embodiment, the invention relates to a process for producing a crystalline antioxidant comprising: (1) providing an aqueous solution comprising a hydrophilic antioxidant; and a polymer selected from hyaluronate-dopamine, alginate-dopamine, chitosan-dopamine, poly(ethylene glycol) dopamine and their derivatives, or mixtures thereof; (2) forming a liquid droplet of the aqueous solution, the droplet having a diameter, wherein polymer-induced nucleation of the antioxidant occurs in the liquid droplets; (3) subjecting the liquid droplet to thermal cycling between a lower temperature and a higher temperature, wherein antioxidant crystal growth occurs at the lower temperature and crystal dissolution occurs at the upper temperature; and maintaining the thermal cycling until the droplet contains one or two antioxidant crystals.

### BRIEF DESCRIPTION OF THE DRAWINGS

FIG. 1 illustrates a scheme of MASC scavenging ROS and preserving the healthy MSC phenotype.

FIG. 2 illustrates CAD image of drop generator geometry.

FIG. 3 illustrates image of drop generation at microfluidic junction.

FIG. 4 illustrates the thermal cycling approach to crystal size distribution control.

FIG. 5 illustrates histograms and representative SEM images of broad CSD crystals and MASC.

FIG. 6 illustrates NAC dissolution profile of broad CSD crystals and MASC.

FIG. 7 illustrates CVbroad/CVMASC release as a function of time.

FIG. 8 illustrates temperature profiles for thermocycling NAC solution drops and optical images of representative drops with crystals at different stages of thermocycling.

FIG. 9 illustrates LIVE/DEAD confocal images of healthy and senescent MSCs.

FIG. 10 illustrates quantification of the fraction of live cells pre- and post-H2O2 exposure.

FIG. 11 illustrates 63x objective confocal images of intracellular oxidative stress and mitochondria in MSCs following H2O2 exposure.

FIG. 12 illustrates representative confocal images of the intracellular ROS and mitochondria for a healthy cell and a senescent cell.

FIG. 13 illustrates box and whisker plots describing the Pearson's colocalization coefficient (r) for healthy and senescent cells.

FIG. 14 illustrates images and quantification of the number of NAC crystals per drop for drops containing no polymer or HA-dopa.

FIG. 15 illustrates the fraction of drops containing a minimum of one crystal (M⁺) over 75 hours for drops containing no polymer drops containing or drops containing 2.5 wt % HA-dopa.

FIG. 16 illustrates the natural log of the fraction of drops containing no crystals as a function of time.

FIG. 17 illustrates fitted homogeneous nucleation rate (k₀), heterogeneous nucleation rate (k), and number of active centers (m) within drops.

FIG. 18 illustrates nucleation efficiency and effect of thermocycling, starting saturation, and drop diameter.

FIG. 19 illustrates histograms and representative SEM images for NAC crystals fabricated and thermal cycled in 25 μm-diameter drops.

FIG. 20 illustrates histograms and representative SEM images for ascorbic acid crystals fabricated and thermal cycled in 25 μm-diameter drops.

FIG. 21 illustrates NAC crystal dissolution.

FIG. 22 illustrates timeline for MSC study.

FIG. 23 illustrates metabolic activity of MSC population following MASC exposure every 2 days for 6 days.

FIG. 24 illustrates confocal images of intracellular ROS.

FIG. 25 illustrates quantified intracellular ROS levels relative to healthy MSCs.

FIG. 26 illustrates confocal images of intracellular oxidative stress in MSCs following no treatment, treatment with bulk-assembled crystals, and treatment with microfluidic-assembled NAC crystals for 6 days.

FIG. 27 illustrates colocalization of intracellular ROS and mitochondria.

FIG. 28 illustrates fraction of cells positively stained for β-galactosidase.

FIG. 29 illustrates β-galactosidase histochemical stain.

FIG. 30 illustrates p16 gene expression normalized to the healthy MSC p16 expression.

FIG. 31 illustrates exosome release relative to baseline exosome release in healthy MSCs.

FIG. 32 illustrates VEGF, IGF, and IL-10 gene expression following 6 days of treatment normalized to the expression in healthy MSCs.

FIG. 33 illustrates CV for bulk crystal and MASC treatment.

FIG. 34 illustrates CVBulk/CVMASC as a function of time for all time-dependent in vitro studies.

FIG. 35 illustrates confocal (oxidative state) and brightfield (B-galactosidase) images of senescent MSCs treated with NAC crystals with a broad CSD or MASC for 6 days.

FIG. 36 illustrates quantification of intracellular ROS in MSCs from confocal images.

FIG. 37 illustrates fraction of MSCs positively stained for β-galactosidase following treatment with NAC crystals with a broad CSD or MASC and normalized gene expression for p16 following treatment with NAC crystals with a broad CSD or MASC.

FIG. 38 illustrates VEGF, IGF, and IL-10-encoding mRNA expression in MSCs following treatment with NAC crystals with a broad CSD or MASC.

FIG. 39 illustrates coefficient of variation for broad CSD crystals and MASC.

FIG. 40 illustrates a schematic of the microfluidic assembly of NAC crystal-loaded microgel beads through a 4-step process: (1) drop generation with a mixture of NAC, HA-dopa, and PEGDA, (2) shear-induced NAC nucleation, (3) NAC crystal growth, and (4) gelation of NAC crystal-loaded PEGDA droplets.

FIG. 41 illustrates shear-induced NAC nucleation in micro-drops produced using microfluidic emulsion generators, with CAD images of microfluidic devices with straight wall channels used for shear-induced ordered mixing and with serpentine channels for shear-induced chaotic mixing. Microdrops of NAC, HA-Dopa, and PEGDA solutions were generated using a single emulsion generator and injected into the two different microfluidic chips for shear-induced mixing

FIG. 42 illustrates shear-induced NAC nucleation in micro-drops produced using microfluidic emulsion generators, where representative flow patterns are shown for ordered and chaotic mixing in drops. The mixing regimes were controlled using straight and serpentine microfluidics, respectively

FIG. 43 illustrates shear-induced NAC nucleation in micro-drops produced using microfluidic emulsion generators, showing images of drops and the quantified fraction of drops containing at least one crystal (M⁺) as a function of flow rate for both ordered and chaotic mixing channels. The residence time was fixed at 10.8 s for all experiments. 250 drops were analyzed for each condition.

FIG. 44 illustrates shear-induced NAC nucleation in micro-drops produced using microfluidic emulsion generators, showing images of drops, and the quantified fraction of drops containing at least one crystal (M⁺) as a function of drop residence time on the chip using the chaotic mixing channel. The flow rate was held constant at 1,000 μL/hr. 250 drops were analyzed for each condition.

FIG. 45 illustrates shear-induced NAC nucleation in micro-drops produced using microfluidic emulsion generators, showing images of drops, and the quantified fraction of drops containing at least one crystal (M⁺) as a function of flow rate using the chaotic mixing channel. The residence time was held constant at 10.8 s. 250 drops were analyzed for each condition.

FIG. 46 illustrates shear-induced NAC nucleation in micro-drops produced using microfluidic emulsion generators, with a CAD image of the microfluidic device with a coupled drop generator and serpentine shear-induced nucleator.

FIG. 47 illustrates shear-induced NAC nucleation in micro-drops produced using microfluidic emulsion generators with a representative image of the microfluidic device with a coupled drop generator and serpentine shear-induced nucleator.

FIG. 48a illustrates gelation of NAC crystal-loaded drops and analysis of NAC release profiles of representative optical images of NAC crystal-loaded PEGDA μgels. The μgel diameter is at 30 μm at a PEGDA wt % range of 20 to 40 wt %.

FIG. 48b illustrates gelation of NAC crystal-loaded drops and analysis of NAC release profiles of representative optical images of NAC crystal-loaded PEGDA μgels. The μgel diameter is at a range of 50 to 70 μm and a PEGDA concentration of 20 wt %.

FIG. 49a illustrates a cumulative release profile of NAC crystal-loaded μgels at 20, 30, or 40% PEGDA wt %.

FIG. 49b illustrates a cumulative release profile of NAC crystal-loaded μgels at 30, 50, or 70 μm-diameter μgels.

FIG. 50a illustrates the half-life of NAC release as a function of PEDGA wt %.

FIG. 50b illustrates the half-life of NAC release as a function of μgel diameter (μm).

### DETAILED DESCRIPTION OF THE INVENTION

In one embodiment, the invention relates to a process for producing a crystalline antioxidant comprising: (1) providing an aqueous solution comprising a hydrophilic antioxidant; and a polymer selected from hyaluronate-dopamine, alginate-dopamine, chitosan-dopamine, poly(ethylene glycol) dopamine and their derivatives, or mixtures thereof; (2) forming a liquid droplet of the aqueous solution, the droplet having a diameter, wherein polymer-induced nucleation of the antioxidant occurs in the liquid droplets; (3) crystallizing the crystalline antioxidant by performing at least one of: (a) subjecting the liquid droplet to thermal cycling between a lower temperature and a higher temperature, wherein antioxidant crystal growth occurs at the lower temperature and crystal dissolution occurs at the upper temperature; and maintaining the thermal cycling until the droplet contains one or two antioxidant crystals, and (b) subjecting the liquid droplet to shear deformation using a shear-induced mixing-on-a-chip device, wherein a probability of nucleation and crystallization of each droplet is increased.

It has unexpectedly been found that such a process provides antioxidants having numerous benefits: (1) increased fraction of droplets containing antioxidant crystals, with (2) lower crystal size distribution (CSD) relative to that obtained in bulk. These features in turn translate to improvements in therapeutic usage: (1) extended release of the antioxidant; (2) improved senescence control; (3) improved scavenging of reactive oxygen species; (4) alleviation of oxidative stress; and (5) reduction in the heterogeneity in mesenchymal stem cells to improve therapeutic efficacy of the MSCs secretome.

The currently claimed process not only results in polymer-stabilized crystallization of the antioxidants, which improves bioactivity, but also avoids the broad crystal size distribution characteristic of conventional processes, which results in an undesirable variation in efficacy. CSD's from bulk crystallization typically are 35+11 μm, while that of the current process typically possesses a much narrower distribution of 13+3 μm. Further, in current techniques that utilize crystallization of antioxidants from droplets, the percentage of drops containing at least one crystal can be as low as 20%. In contrast, the currently described process increases the fraction of droplets having at least one crystal to above 86%.

### Hydrophilic Antioxidants

In the process of the current subject matter, antioxidants crystallized from an aqueous solution with stabilizing polymers has been found to improve release kinetics and efficacy. Preferably, the hydrophilic antioxidant is selected from N-acetylcysteine, ascorbic acid, vitamin C, uric acid, and glutathione. More preferably, the hydrophilic antioxidant is N-acetylcysteine and ascorbic acid. Preferably, the concentration of the hydrophilic antioxidant in the aqueous solution is from 200 mg/mL to 800 mg/mL. More preferably, the concentration of the hydrophilic antioxidant in the aqueous solution is from 300 mg/mL to 400 mg/mL.

### Stabilizing Polymers

The stabilizing polymer utilized in the process of the current subject matter is selected from hyaluronate-dopamine, alginate-dopamine, chitosan-dopamine, poly(ethylene glycol) dopamine and their derivatives, or mixtures thereof. Preferably, the stabilizing polymer is hyaluronate-dopamine.

Preferably, the concentration of the stabilizing polymer in the aqueous solution is from 5 mg/mL to 80 mg/mL. More preferably, the concentration of the polymer in the aqueous solution is from 15 mg/mL to 20 mg/mL.

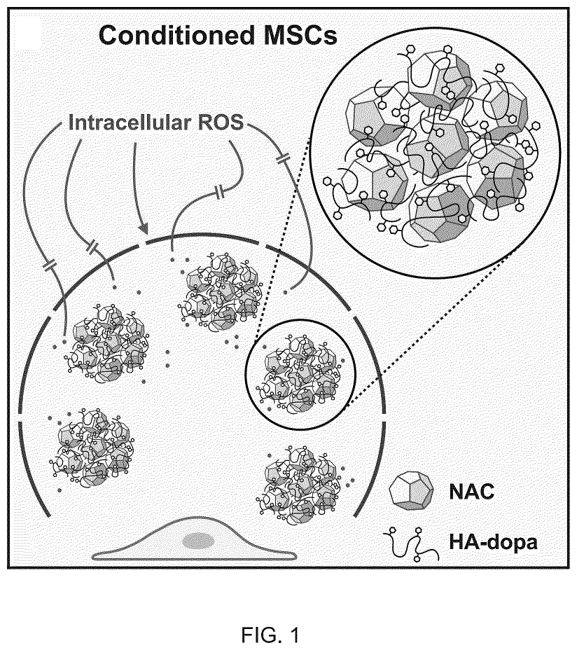

As a model API-polymeric excipient system, antioxidizing NAC crystals stabilized by hyaluronatedopamine (HA-dopa) polymers were fabricated. It is believed that a reduction in the CSD will further extend NAC release and minimize variation through more uniform aggregation. The resultant MASC can then actively scavenge ROS, alleviate oxidative stress, and reduce the heterogeneity in MSCs in order to improve the therapeutic efficacy of the MSCs secretome (FIG. 1).

### Formation of Liquid Droplets

Compared to crystallization in batch, drop-microfluidic techniques have the benefit of precise compartmentalization. It has been found that drug loading in drops is highly uniform, and, provides nucleation control by avoiding reactor-induced nucleation.

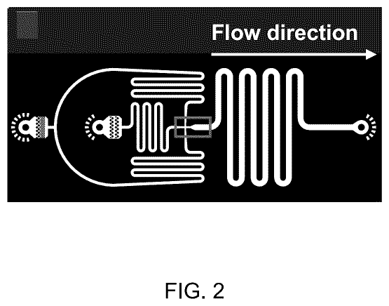

After preparation of an aqueous solution containing hydrophilic antioxidant and polymer, liquid droplets are formed. The liquid droplets having a droplet diameter from 10 to 200 μm are generated in commercially available drop generator equipment, as shown schematically in FIG. 2. Preferably, the droplet diameter is from 10 to 25 μm.

### Thermal Cycling of Liquid Droplets

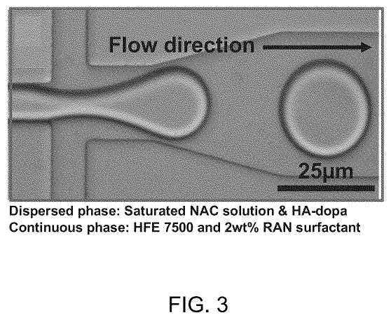

Historically, a drawback to the use of liquid droplets as described above is that over reasonable time scales, the fraction of microdroplets containing at least one hydrophilic API crystal can be as low as 20%, challenging both high-throughput operation and material recovery. The method of the current subject matter utilizes thermal cycling to improve performance. The thermal cycling is continued until one or two crystals remain in the droplet. Preferably, the droplet contains a single crystal as shown in FIG. 3. Preferably, the thermal cycling is completed within a time of 12 to 24 hours.

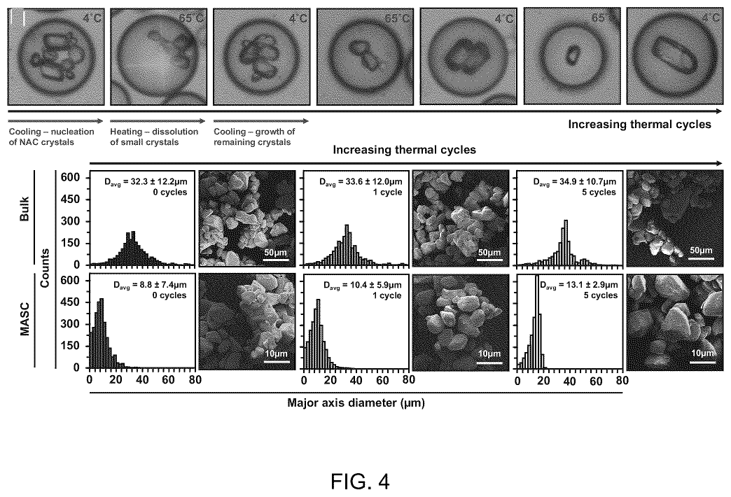

The thermal cycling of the liquid droplets promotes larger crystals having a narrow CSD, which maximizes the efficacy variation among the antioxidant crystals produced; e.g., a reduction in the CSD will extend release of the hydrophilic antioxidant with minimal variation through more uniform aggregation. During thermal cycling, the droplet heating events fully dissolve small crystals in the crystal population. As such, during the next cooling step, growth is preferred on the larger crystals that survived the prior heating step over the formation of nuclei, as shown in (FIG. 4). At the end of the thermal cycling, (ending on the low temperature), the temperature is held for a final hold time. Preferably, the final hold temperature is from 4° C. to 10° C. Preferably, the final hold time is 10-14 hours.

Preferably, the lower temperature of the thermal cycling is from 4° C. to 25° C. Preferably, the higher temperature of the thermal cycling is from 45° C. to 65° C. At both the lower temperature and the higher temperature portions of each thermal cycle, the temperature is preferably maintained for a time period of 20 to 60 minutes at the low temperature, and 10 to 30 minutes at the lower temperature.

Preferably, the temperature difference between the lower temperature and the higher temperature is from 20° C. to 60° C.

Preferably, the total number of cycles in the thermal cycling is 3 to 7. For example, if a total of three thermal cycles were necessary, the cycle steps would be as follows: high temp1-lowtemp1-hightemp2-lowtemp2-hightemp3-lowtemp3. As mentioned above, preferably a final hold time would follow the final step of the thermal cycling, where the droplet would be held at a final hold temperature of 4° C. to 10° C. The purpose of the final hold time is to ensure all NAC has fully crystallized.

### Shearing of Liquid Droplets Using a Shear-Induced Mixing-On-a-Chip Device

Historically, a drawback to the use of liquid droplets as described above is that over reasonable time scales, the fraction of microdroplets containing at least one hydrophilic API crystal can be as low as 20%, challenging both high-throughput operation and material recovery. The method of the current subject matter can also utilize shear-induced crystallization through serpentine microfluidics directly connected with a microdroplet (or drop) generator. Such equipment is known as a shear-induced mixing-on-chip device. This design is inspired by the finding that increased nucleation kinetics can result from prenuclei cluster collisions and the formation of larger clusters, which shorten the crystal induction time. The straight and serpentine channels were used to identify the optimal device geometry for shear-induced crystallization. The resulting shear force at the interface of the drop and the narrowed channel walls can induce mixing in the drops, where the channel geometry can control the degree of mixing. In particular, the serpentine channel can cause chaotic mixing, while the straight channel makes an ordered orbital flow pattern. In this design the chip aids mixing of the molecules in the individual drops. Preferably, the shear-induced crystallization is completed within a time of 1 minute.

This shear-induced method enables the continuous fabrication of uniform microgel particles encapsulating antioxidant crystals with minimal size variation. By integrating a microdroplet generator, serpentine microfluidics for shear-induced crystallization, and a curing unit (e.g., an ultraviolet source), it is possible to produce novel microparticles capable of releasing molecular cargos over a week in under a minute. Unlike methods that rely on heating and cooling for drug crystallization, this approach eliminates the need for thermal cycling, making it suitable for manipulating the crystallinity of heat-sensitive biomacromolecules, such as peptides and proteins.

The shear force on the liquid droplets in this method promotes larger crystals, which maximizes the efficacious variation among the antioxidant crystals produced; e.g., the crystallization and further encapsulation in microgels will extend the release of the hydrophilic antioxidant. During shear deformation, the drug molecules in microdroplets can align in one direction, facilitating more effective collisions, while preventing supersaturation gradients. Thus, shear reduces the energy barrier for nucleation, promoting the formation of a critical nucleus necessary for crystallization. At the end of the shear deformation, the microdroplets are heated or exposed to ultraviolet light to activate gelation within microdroplets.

Preferably, the flow rate difference between the lower flow rate and the higher flow rate is from 100 to 1,000 μL/hr.

Preferably, the microdroplet (or drop) generator and the source of ultraviolet light are connected to the serpentine microfluidics for the continuous process.

### EXAMPLES

The following Examples further detail and explain the preparation and performance of the inventive microcrystal assembly for senescence control, i.e., specially prepared antioxidant crystals. Those skilled in the art will recognize many variations that are within the spirit of the invention and scope of the claims.

### Fabrication of Single Emulsion Drop Generator

SU-8 2025 was used to fabricate microfluidic devices with 30 μm channel heights, and SU-8 2050 was used to fabricate devices with 50 μm channel heights. Silicon wafers were cleaned with IPA, DI water, and acetone, and dried with N₂ at 65° C. prior to spin coating of SU-8. According to the supplier's recommendations, SU-8 was spin-coated onto the Si wafers, soft baked, hard baked, and exposed to UV. Photomasks of device features were designed in AutoCAD and produced by CADart. Following exposure, the wafers underwent a postexposure bake and development with Kayaku Advanced Materials SU-8 developer according to the supplier's recommendations. A 1:10 weight ratio of PDMS monomer and curing agent was poured into the SU-8 stamps and degassed for several hours. The PDMS was transferred to an oven at 65° C. and cured overnight. The PDMS was removed and bound to glass following plasma treatment of the surface of the glass slide and PDMS. The device was transferred to an oven at 65° C. to allow the bond to set. To increase the hydrophobicity of the PDMS channels, aquapel was injected into the device and dried with N₂ prior to drop making.

### Synthesis of Hyaluronate-Dopamine (HA-Dopa)

HA (hyaluronate) was dissolved in a 0.1 m solution of MES (pH=5) for 12 h at room temperature. Once the polymer was fully dissolved, 1-Ethyl-3-(3-dimethylaminopropyl) carbodiimide (EDC) and N-Hydroxysulfosuccinimide (sulfo-NHS) were dissolved in the solution for 30 min. EDC and sulfo-NHS were both added in excess at a 0.625:1 molar ratio to sodium glucuronate of HA. Dopamine hydrochloride was added at the appropriate molar ratio. The mixture was dialyzed against DI water using a dialysis tube (MWCO=3.5 kDa) for 2 days and then lyophilized for 3 days before storage at 4° C. until future use. The degree of substitution of dopamine (DSdopa) was controlled by fixing the molar ratio between dopamine to sodium glucuronate of HA at 8:9. This ratio leads to ˜20% of the carboxylic acids on HA being conjugated with dopamine.

### Bulk Fabrication of Crystals

50 mg (5% w/w of NAC) of polymer additives (HA or HA-dopa) were dissolved in DI water (10 mL) for 6 h at 45° C. to ensure the complete dissolution. Then, 1 g of NAC was added into the polymer solution and stirred at 45° C. until all the solids were dissolved (6 h). The solution was cooled to 4° C. at a rate of 1° C. min⁻¹ and heated to 65° C. at a rate of 1° C. min 1 for anywhere between 0 and 5 thermocycles, and finally cooled to 4° C., where the temperature was held for an additional 12 h with under constant stirring. The obtained crystal solution was filtered through a polyvinylidene fluoride (PVDF) membrane (HVLP04700, pore size: 0.45 μm, Millipore) to collect NAC crystals. The crystals were gently washed 2-3 times with DI water to remove the unbound polymers. Then, the samples were dried under vacuum at room temperature for 2 days. NAC crystals were stored at room temperature under N₂ until used for future experiments. The morphology of NAC crystals was examined by an optical microscope (Leica DMIL) and an environmental scanning electron microscope (ESEM, Quanta FEG 450, FEI) at 7 kV acceleration voltage.

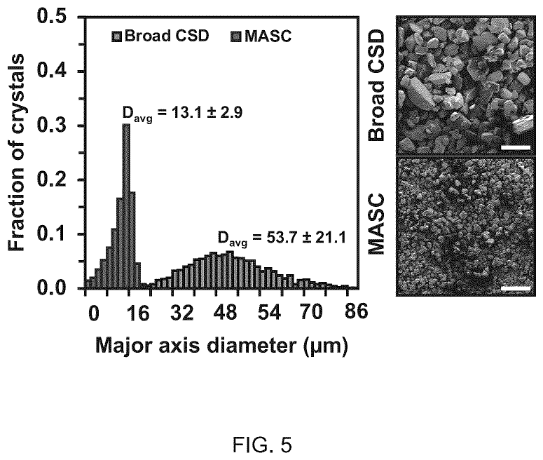

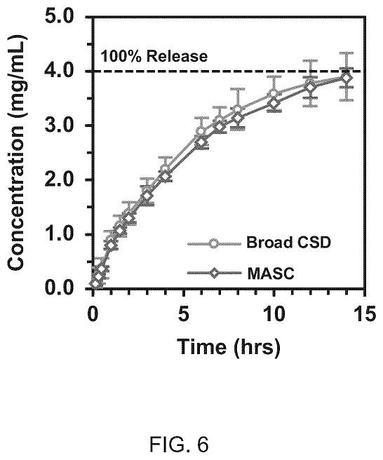

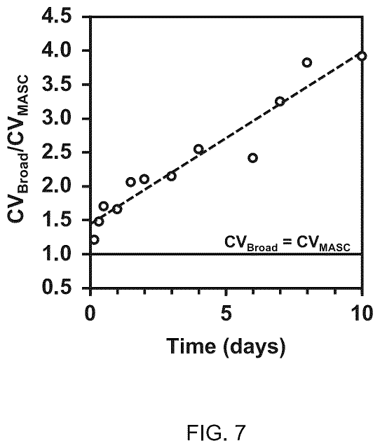

In order to illustrate that efficacy differences between the bulk assembled NAC crystals and microfluidic-assembled NAC crystals are coupled to the release variation, a second bulk-assembled crystal (“Broad CSD crystal”) was engineered. The bulk-assembly followed the same protocol previously described, except an additional crystal growth step was employed. Following the 5 thermocycles and 12 h hold at 4° C., 200 μL of supersaturated NAC (20 mg of NAC was dissolved in 200 μL at RT and slowly cooled to 4° C.-0.1° C. min 1) was added to the crystal suspension. Rather than the formation of new nuclei, crystal growth occurred on the current crystal population. This procedure resulted in larger crystals that dissolve slower but have the same CSD as the first bulk-assembled crystals (FIGS. 5 and 6). Again, the release variation was higher for this crystal population than for the microfluidic crystals (FIG. 7). The obtained crystal solution was filtered through a PVDF membrane (HVLP04700, pore size: 0.45 μm, Millipore) to collect NAC crystals. The crystals were gently washed 2-3 times with DI water to remove the unbound polymers. Then, the samples were dried under vacuum at room temperature for 2 days. NAC crystals were stored at room temperature under N₂ until used for future experiments. The morphology of the NAC crystals was examined by an optical microscope (Leica DMIL) and an environmental scanning electron microscope (ESEM, Quanta FEG 450, FEI) at 7 kV acceleration voltage.

### Microfluidic Assembly of Crystals

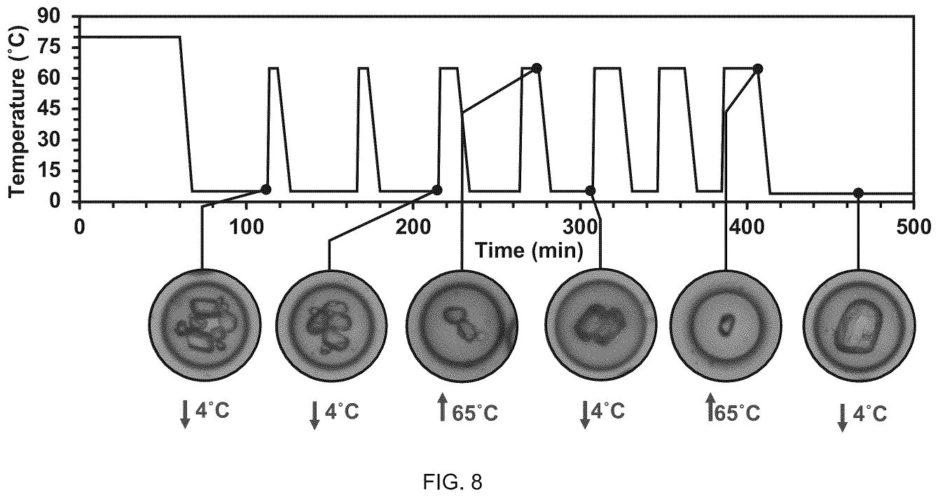

364 mg of NAC and 18.2 mg (5% w/w of NAC) of polymer additives (HA or HA-dopa) were dissolved in DI water (1 mL) for 6 h at 55° C. to ensure complete dissolution. Using a heated glass syringe (heated with a conductive fabric (8.4 V) to 55° C.), the NAC solution was injected into the microfluidic device (Q=100 μL h−1) while HFE 7500 with 2 wt % RAN surfactant was injected into the second inlet of the microfluidic device (Q=200 μL h−1) to generate drops with a diameter of 25 μm. Although the NAC saturation temperature was 45° C., the operating temperature of 55° C. was chosen to prevent crystallization prior to drop generation as the chip itself was not heated. The drop diameter could be changed for subsequent experiments by changing the width of the aqueous-organic (drop-making) junction or by changing the ratio of the aqueous and organic phase flow rates. Drops were collected in PCR tubes and topped with a mineral oil layer to minimize evaporation and shrinking of the aqueous drops. Using a thermocycler (BIO-RAD T100 thermocycler), the temperature of the drops was brought to 80° C. for 1 h to ensure any crystals/nuclei dissolved. Then drops were cycled (up to 7 times) between 65 and 5° C., and held at 4° C. for 24 h after thermal cycling. Heating and cooling rates, and hold times are shown in FIG. 8. Following crystallization, the drops were washed with 10% perfluorooctanoic acid in HFE7500 to break drops. The obtained crystal solution was filtered through a polyvinylidene fluoride (PVDF) membrane (HVLP04700, pore size: 0.45 μm, Millipore) to collect NAC crystals. The crystals were gently washed 2-3 times with DI water to remove the unbound polymers. Then, the samples were dried under vacuum at room temperature for 2 days. NAC crystals were stored at room temperature under N₂ until used for future experiments. The morphology of NAC crystals was examined by an optical microscope (Leica DMIL) and an environmental scanning electron microscope (ESEM, Quanta FEG 450, FEI) at 7 kV acceleration voltage.

### Post-Crystallization Processing

During the thermocycling, drop merging occurred resulting in crystals with significantly larger major axis diameters. As such, density gradient centrifugation was employed to separate drop size. Following thermocycling, drop suspensions were lightly centrifuged (800 rpm) for 45 s. For analysis of crystal diameters, only the bottom ⅓ of drops were collected and washed with 10% perfluorooctanoic acid to harvest the NAC crystals. While the drop diameter can be used to control crystal size, another approach is to add heating steps post-thermal cycling to partially dissolve crystals but preserve the CSD. This approach helps to maximize the crystallization efficiency by having a larger driving force for crystallization (45->4° C. compared to 45->35° C.). Following 5 thermocycles in 25 μm diameter drops as previously described, the temperature of the drops was raised to either RT or 35° C. and held at this temperature for 6 h. The drops were then washed with HFE containing 10% perfluorooctanoic acid to harvest the NAC crystals. The obtained crystal solution was filtered through a PVDF membrane (HVLP04700, pore size: 0.45 μm, Millipore) to collect NAC crystals. The crystals were gently washed 2-3 times with DI water to remove the unbound polymers. Then, the samples were dried under vacuum at room temperature for 2 days. NAC crystals were stored at room temperature under N₂ until used for future experiments. The morphology of NAC crystals was examined by an optical microscope (Leica DMIL) and an environmental scanning electron microscope (ESEM, Quanta FEG 450, FEI) at 7 kV acceleration voltage.

### Characterization of NAC Dissolution

50 mg of NAC crystals were placed in a dialysis tube (MWCO=500-1000 Da) with phosphate buffered saline (PBS, 1 mL). The dialysis tube was placed in the 499 mL of PBS media and incubated at 37° C. under continuous shaking at100 rpm. At the designated time points, dissolved NAC was collected from the incubation media and determined by reading the absorbance at the wavelength of 260 nm using the microplate spectrophotometer (Infinite 200 PRO, Tecan).

### Human Adipose-Derived Mesenchymal Stem Cells (MSCs) Culture and Treatment

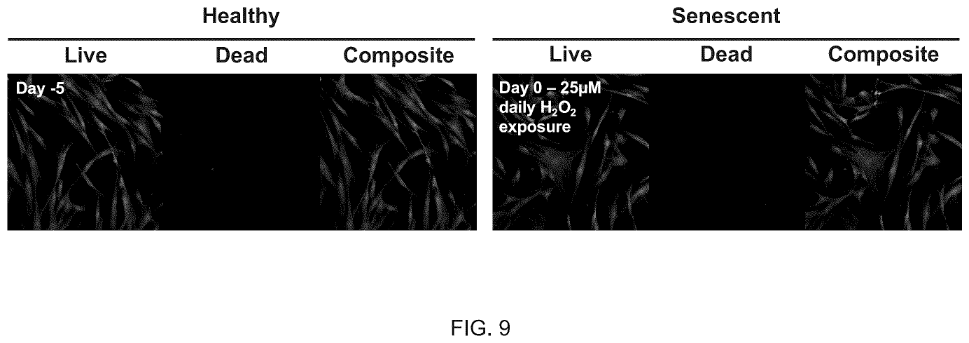

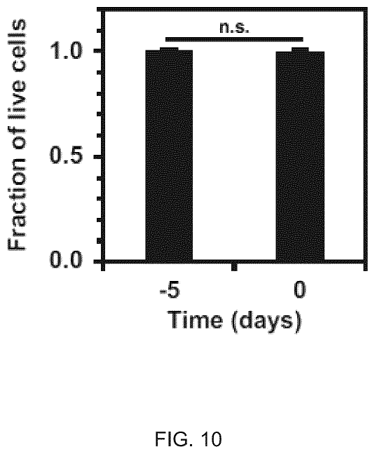

In order to put MSCs into a senescent state, cells were exposed to H2O2 to disrupt mitochondrial functions and upregulate intracellular ROS levels. First, MSCs (passage 2) were seeded at 25% confluency on 0.1% gelatin-coated glass bottom dishes and allowed to proliferate to 50% confluency over 24 h. After 24 h, the fetal bovine serum (FBS) concentration was reduced from 10% to 5% to slow proliferation, and 25 μm of H2O2 was added to the media daily for 5 days. To confirm that the H2O2 treatment did not cause cell death, a LIVE/DEAD assay was performed according to the supplier's recommendations. 2 μm calcein AM (marker for live cells—green fluorescence) and 4 μm EthD-1 solution (marker for dead cells-red fluorescence) were added directly to the cells following H2O2 exposure and washing with PBS and allowed to incubate for 45 min. Finally, the cells were imaged using a confocal microscope (Zeiss LSM 700), and the intensity was quantified using ImageJ software (NIH) (FIG. 9). The assay was used to assess MSCs either cultured with H2O2-free media or exposed to 25 μm H2O2 daily for 5 days (FIG. 10).

To confirm the biocompatibility and toxicity of MASC, a commercially available MTT assay kit (11 465 007 001, Roche) was used to measure the metabolic activity of MSCs exposed to MASC at a concentration range of 50-500 μm. The assay was performed with a few modifications to the manufacturer's directions. MSCs were seeded into wells in a 96-well plate at a density of 10 000 cells per well. When the cells reached 50% confluency, the FBS concentration was reduced from 10% to 5% to slow proliferation. MSCs were exposed to MASC every 2 days alongside the media change for 6 days. After the exposure period, 90 μL of MASC-free media and 10 μL of MTT solution was used to replace media. The cells were incubated at 37° C. for 3 h. The formazan crystals formed as result of metabolic cell function were dissolved with 65 μL of DMSO. The absorbance of MTT was measured using a microplate spectrophotometer (Infinite 200 Pro, Tecan) at the wavelengths 550 and 650 nm. The relative metabolic activity was quantified as [(A550-A650) MASC/(A550-A650) Control]. Five samples (n=5) were prepared for each condition. Following H2O2 exposure, the MSCs were treated with either NAC-free media, bulk-assembled NAC crystals (10 ppm), or microfluidic assembled NAC crystals (10 ppm) every 2 days for 6 days.

### Intracellular Reactive Oxygen Species (ROS) Levels in MSCs

MSCs were seeded 0.1% gelatin-coated 18 mm cover glass at 10 000 cells per dish exposed to H2O2 as described before. After the H2O2 exposure, the cells were treated with either NAC-free media, bulk-assembled NAC crystals (10 ppm), or microfluidic-assembled NAC crystals (10 ppm) every 2 days for 6 days. Following treatment, cells were washed 3 times with 1x PBS. After 2, 4, or 6 days, the intracellular oxidative stress was evaluated using the CellROX green reagent (ThermoFisher), following the manufacturer's instruction. Briefly, the MSCs were incubated with 5 μm of CellROX reagent for 30 min, and the cells were washed with 1x PBS three times. To identify the mitochondria, cells were stained with 300 nm of MitoTracker Red (ThermoFisher) for 45 min. The cells were washed with 1xPBS three times. The cells were fixed with a 1:1 acetone: methanol solution for 15 min. For the nuclear counterstain, cells were stained with 500 nm of 4′,6-diamidino-2-phenylindole (DAPI). Finally, the cells were imaged using a confocal microscope (Zeiss LSM 700), and the intensity was quantified using ImageJ software (NIH). 5 samples (n=5) were prepared for each condition.

### Senescence Characterization in MSCs

The histological kit for β-galactosidase detection was used to determine if a cell is in a senescent state. MSCs were exposed to H2O2 as previously described for 5 days, then washed with 1x PBS 5 times to remove any extracellular H2O2. The cells were then treated with either NAC-free media or NAC crystals according to the previously described protocol. Following treatment, the cells were fixed with the supplied fixation buffer for 7 min at room temperature and washed with 1x PBS 3 times. According to the supplier's recommendations, the staining mixture was prepared and used to cover the cells. The cells were incubated in the staining mixture at 37° C. without CO2 for 8 h. The CO2 levels were minimized to ensure no fluctuations in the pH levels. Cells were washed with 1x PBS 3 times and imaged with an optical microscope (Leica DMIL). 5 samples (n=5) were prepared for each condition. As a second marker for senescence, the gene expression for the tumor suppression protein, p16, was quantified via qRT-PCR. MSCs were exposed to 25 μm H2O2 daily for 5 days and then treated with either NAC-free media, bulk-assembled NAC crystals (10 ppm), or microfluidic-assembled NAC crystals (10 ppm) every 2 for 6 days.

Following treatment, cells were washed 3 times with 1×PBS, detached with trypsin, and centrifuged at 1250 rpm for 5 min. The supernatant was discarded, and cells were lysed and homogenized in the supplied RLT Plus buffer. Following the supplier's recommendations, the cell debris and DNA were isolated from the RNA through centrifugation with the supplied spin columns and filters. The harvested RNA was then synthesized into cDNA using the iScript cDNA Synthesis Kit.

Following the supplier's recommendations, 4 μL of the iScript Reaction Mix, 1 μL of iScript Reverse Transcriptase, and 15 μL of total RNA in nuclease-free water (0.05 μg μL−1) were added to dome-capped PCR tubes and thermal cycled (BIO-RAD T100 thermocycler) according to the supplier's recommendations. Using iTaq Universal SYBR Green Supermix according to the supplier's recommendations, the forward and reverse primers for p16 (designed and ordered from Integrated DNA Technologies) were mixed with the synthesized cDNA in a PCR tube via vortexing and added to a PCR plate. qRT-PCR was used to quantify (2-AACT method) the expression of p16 and was normalized to GAPDH gene expression levels. 5 samples (n=5) were prepared for each condition.

### Quantification of Exosome Release and Paracrine Gene Expression in MSCs

The exosomes secreted from the MSCs (˜30-120 nm) were isolated from the cell media daily using Total Exosome Isolation Reagent (ThermoFisher). As previously described, MSCs were exposed to 25 μm H2O2 daily for 5 days and then treated with either NAC-free media, bulk-assembled NAC crystals (10 ppm), or microfluidic-assembled NAC crystals (10 ppm) every 2 days for 6 days. At the end of each day, 50% of the cell media was harvested and centrifuged at 2000×g for 30 min to remove any cell debris or crystals. The supernatant was transferred to a separate centrifuge tube and mixed with the supplied reagent (1:2 v/v), vortexed, and incubated at 4° C. overnight. After incubation, the sample was centrifuged at 10 000× g for 60 min at 4° C. The supernatant was discarded, and the exosomes were resuspended in 1x PBS. The concentration of the exosomes was calculated using nanoparticle tracking analysis (NanoSight NS300).

Expression levels of mRNAs encoding paracrine factors including VEGF, IGF, and IL-10, were quantified via qRT-PCR. MSCs were exposed to 25 μm H2O2 daily for 5 days and then treated with either NAC free media, bulk-assembled NAC crystals (10 ppm), or microfluidic-assembled NAC crystals (10 ppm) every 2 days for 6 days. Following treatment, cells were washed 3 times with 1×PBS, detached with trypsin and centrifuged at 1250 rpm for 5 min. The supernatant was discarded, and cells were lysed and homogenized in the supplied RLT Plus buffer. Following the supplier's recommendations, the cell debris and DNA were isolated from the RNA through centrifugation with the supplied spin columns and filters. The harvested RNA was then synthesized into cDNA using the iScript cDNA Synthesis Kit. Following the supplier's recommendations, 4 μL of the iScript Reaction Mix, 1 μL of iScript Reverse Transcriptase, and 15 μL of total RNA in nuclease-free water (0.05 μg μL-1) were added to dome-capped PCR tubes and thermal cycled (BIO-RAD T100 thermocycler) according to the supplier's recommendations. Using iTaq Universal SYBR Green Supermix according to the supplier's recommendations, the forward and reverse primers for p16 (designed and ordered from Integrated DNA Technologies) were mixed with the synthesized cDNA in a PCR tube via vortexing and added to a PCR plate. qRT-PCR was used to quantify the expression (2-AACT method) of VEGF, IGF, and IL-10, and was normalized to GAPDH gene expression levels. 5 samples (n=5) were prepared for each condition.

### Coefficient of Variation Calculation and Statistical Analysis

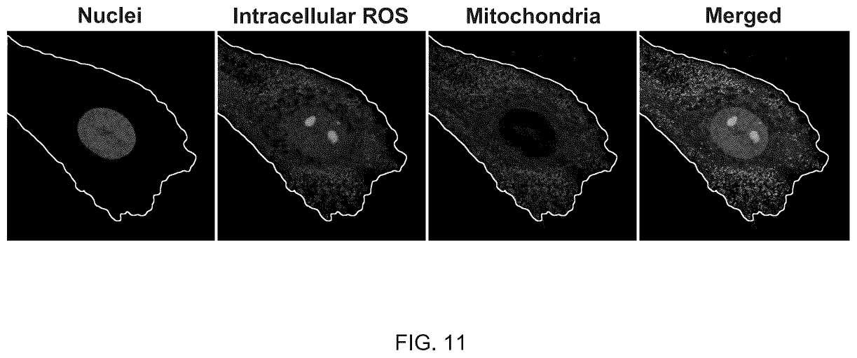

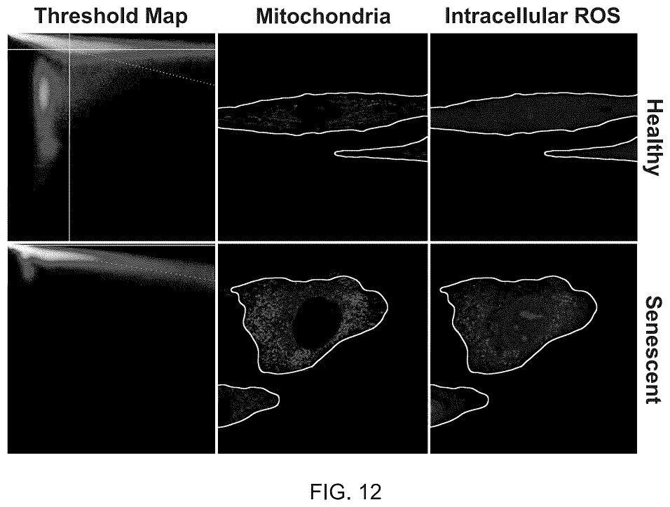

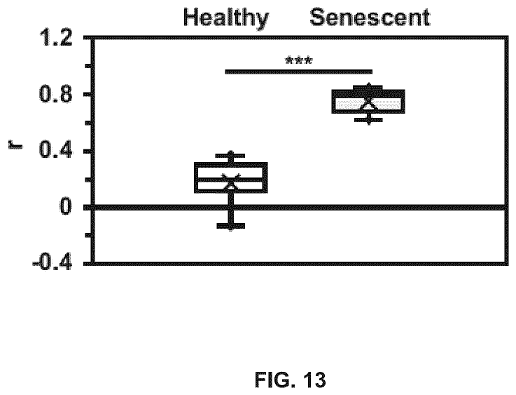

The coefficient of variation (CV) was calculated as the standard deviation over the mean. The CV was used as a metric to determine the variability of each sample condition over the time course of an experiment. The CV was calculated for all experiments involving either the bulk-assembled NAC crystals or the microfluidic-assembled NAC crystals. To determine the colocalization of the mitochondria and intracellular ROS, the built-in tool (Coloc2) in ImageJ (NIH) was used to determine the Pearson's coefficient, a metric for colocalization that takes into account relative pixel intensity (FIGS. 11-12 and FIG. 13). 10 samples (n=10) were prepared for each condition.

### Example 1. HA-Dopa Induced Nucleation of N-Acetylcysteine in Drops

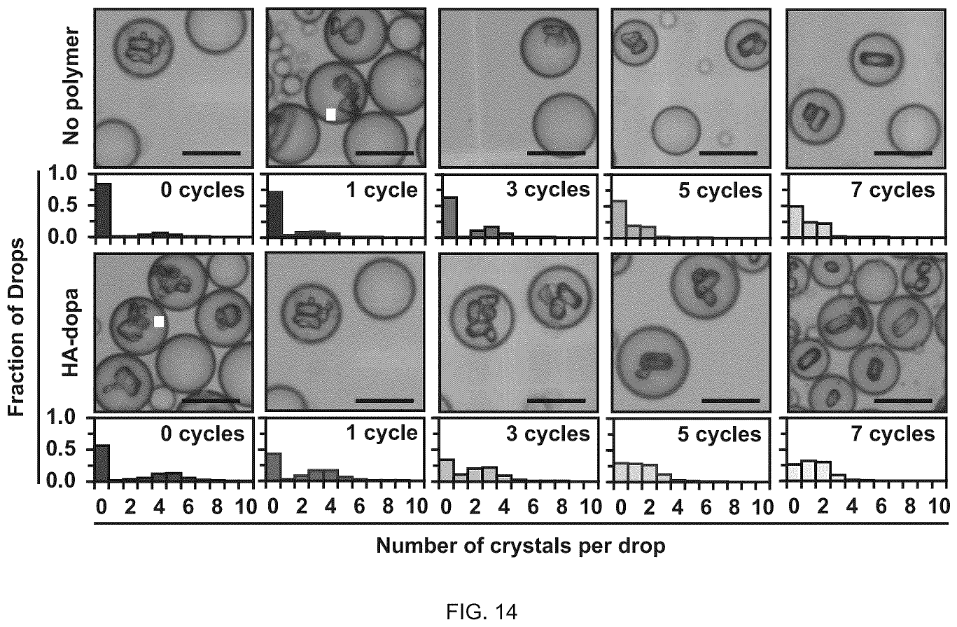

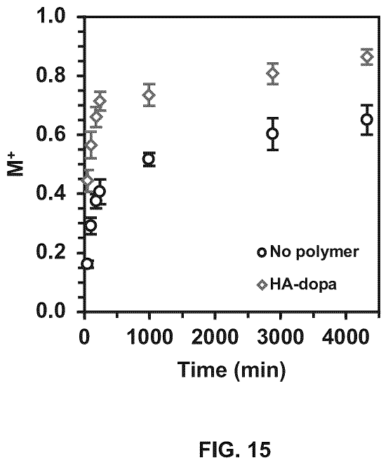

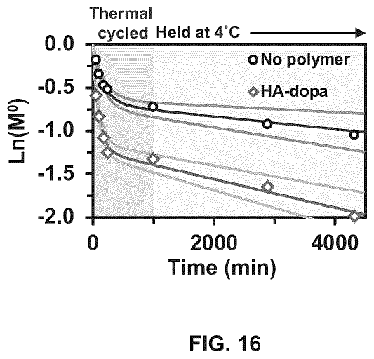

To study if HA-dopa loading in drops can improve the fraction of drops containing crystals, the number of crystals per drop as a function of drop content (no polymer or HA-dopa) was quantified (FIG. 14). During thermal cycling, the fraction of drops containing no crystals (M0) was less for drops with HA-dopa. After 7 thermal cycles and a 24 h hold time to reach complete crystallization, 86.3% of drops containing HAdopa had at least 1 crystal (M⁺), a 4-fold improvement than previously reported efficiencies for similar cooling crystallization timescales (FIG. 15). Furthermore, the time for 50% of drops to contain a crystal is 10-fold faster for HA-dopa loaded drops, demonstrating the drastic effect of HA-dopa on early nucleation kinetics and considerably improving the practicality of crystallization using droplet microfluidics. The high nonlinearity of In (M0) suggests that more than one type of nucleation is occurring (FIG. 16). Therefore, we modeled the primary nucleation with a probabilistic model proposed by Pound and LaMer. With this model, the homogeneous (k0) was decoupled from heterogeneous (k) nucleation rates and predicted the mean number of active nucleation centers (m) (Equation (1)).

M⁰=e^{-m}(e^{-k0t}−1)+e^{-m}(me^{-kt}) (1)

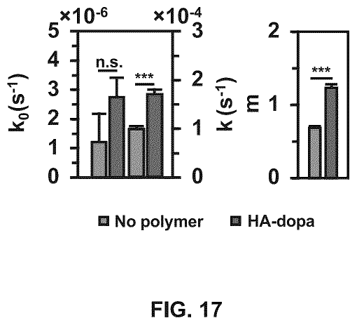

Heterogeneous nucleation was dominant in both drop conditions, as evidenced by k being over an order of magnitude greater than k0. Nonetheless, k and m were greatly increased in drops containing HA-dopa (FIG. 17). As such, the observed increase in nucleation kinetics in HA-dopa loaded drops is attributed to the promotion of heterogeneous nucleation and nuclei stabilization facilitated by polymer adsorption.

### Example 2. Crystal Size Distribution Control with In-Drop Thermal Cycling

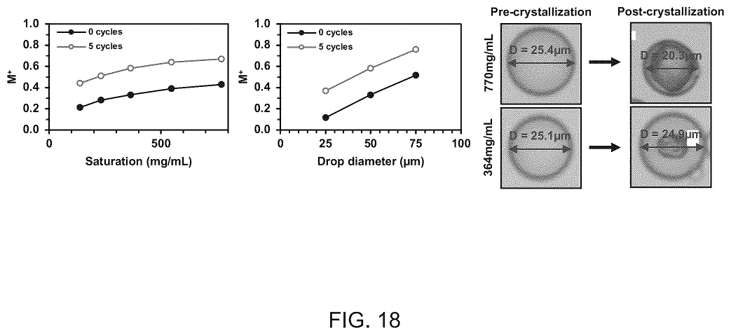

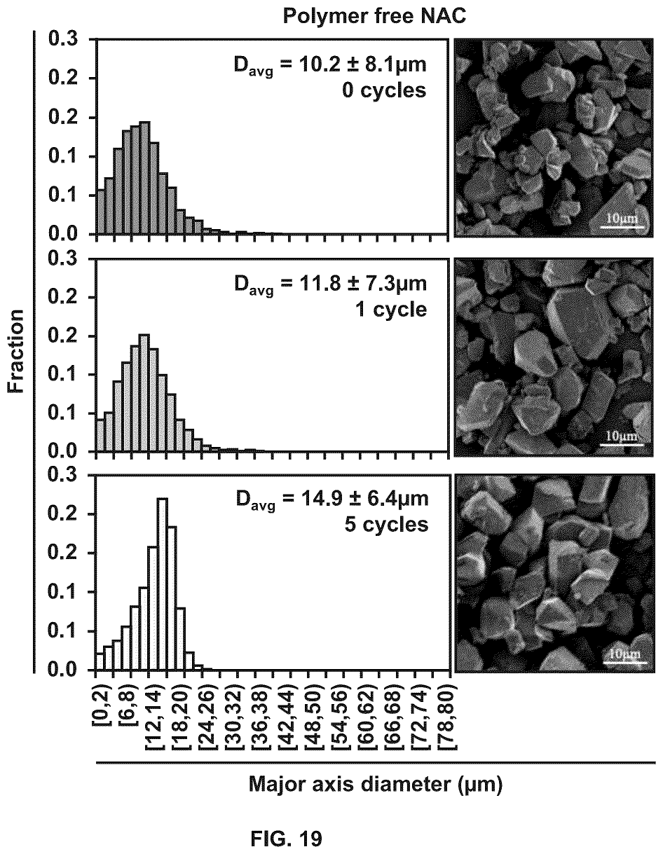

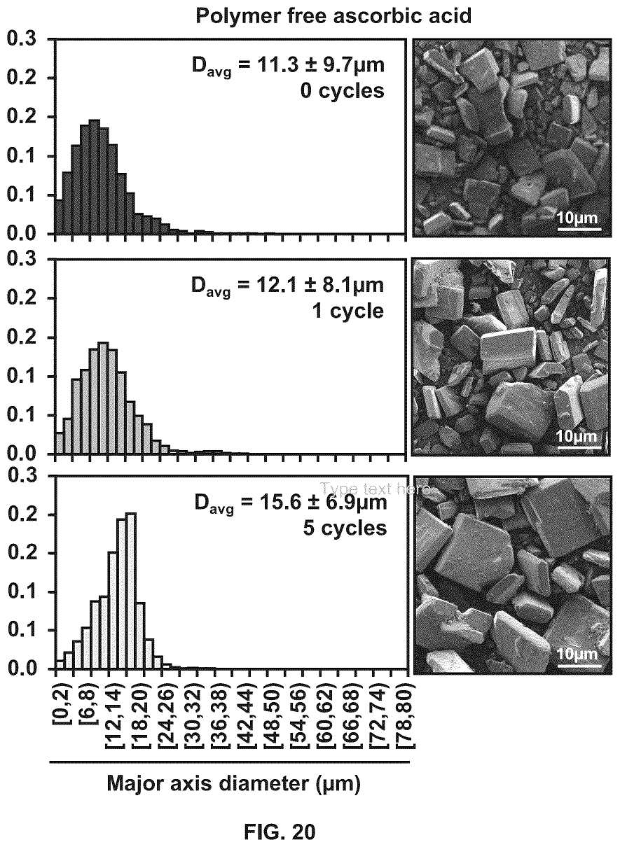

Bulk and microfluidic fabrication approaches were compared to illustrate the extent that controlling CSDs minimize NAC release variation. For NAC crystallization, the supersaturation condition (364 mg mL-1 at 45° C.) was chosen that provided a large nucleation driving force and prevented crystal-induced drop deformation (FIG. 18,). In order to illustrate the ability to fabricate crystals small enough for various delivery routes (˜10 μm), 25 μm diameter drops were generated that were collected in PCR tubes. The collected drops were then thermal cycled 5-times between 65 and 5° C., followed by a 24 h hold time at 4° C. In principle, during thermal cycling, heating events fully dissolve small crystals in the crystal population. As such, during the next cooling step, growth is preferred on the larger crystals that survived the aforementioned heating step over the formation of nuclei (FIG. 4). Crystallization without HA-dopa was also explored (FIG. 19). To emphasize process compatibility with other hydrophilic APIs, ascorbic acid in-drop was crystallized and thermal cycled (FIG. 20). Separately, bulk-assembled NAC crystals (denoted as “bulk”) with HA-dopa were processed with the same thermal cycling conditions. The histograms quantifying the major axis diameter of NAC crystals illustrate two significant differences between the fabrication approaches: 1) droplet confinement reduced the average crystal diameter (13.1 μm compared to 34.9 μm in bulk), and 2) thermal cycling in drops narrowed the CSD (7.4-2.9 μm). In contrast, bulk thermal cycling did not reduce the CSD, likely due to uncontrollable secondary nucleation.

### Example 3. N-Acetylcysteine Crystal Dissolution

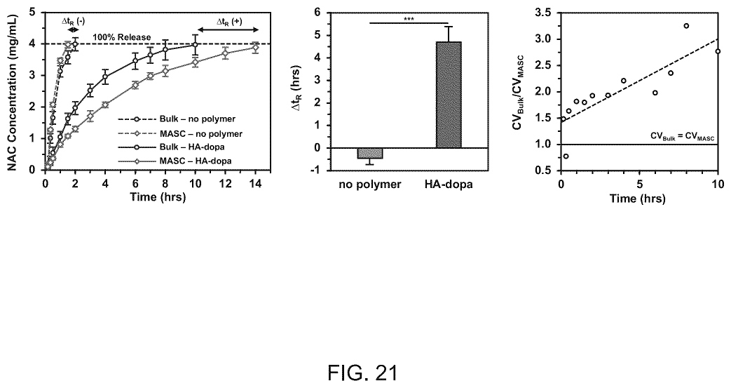

The dissolution profile of NAC crystals and the release variation were quantified (FIG. 21). Surprisingly, despite the smaller size, microfluidic-assembled NAC crystals extended the release of NAC by 40% compared to bulk NAC crystals. For the HA-dopa NAC crystal system, reducing the CSD considerably enhances the stabilizing effect of HA-dopa on the NAC crystals. Furthermore, the release variation, quantified as the ratio of the coefficient of variation (CV) was greater than 1.0 for all but one time point, and increased with time, illustrating faster propagation of release variation for the crystal populations with a broader CSD.

### Example 4. Efficacy of MASC to Protect Against Senescence in MSCs

### Characterization of MSCs Oxidative State

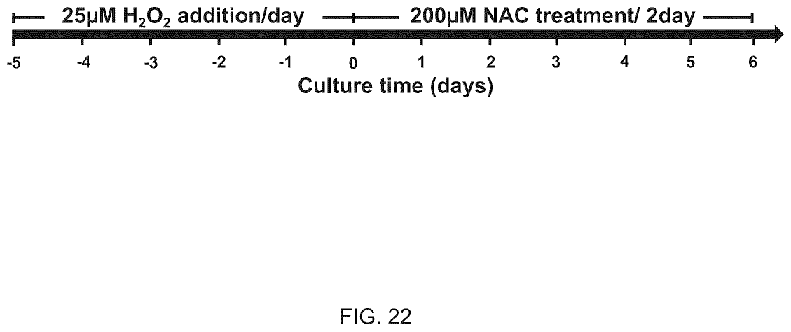

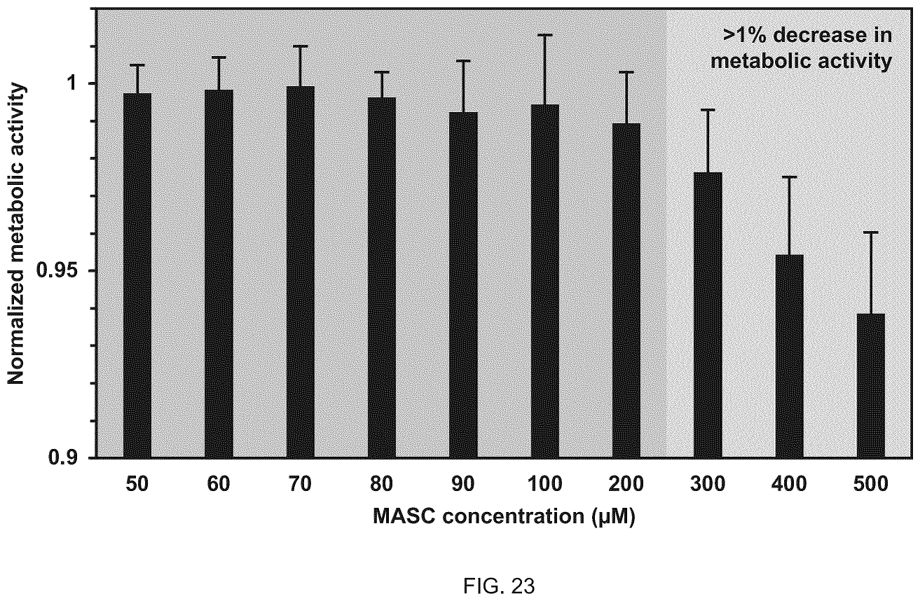

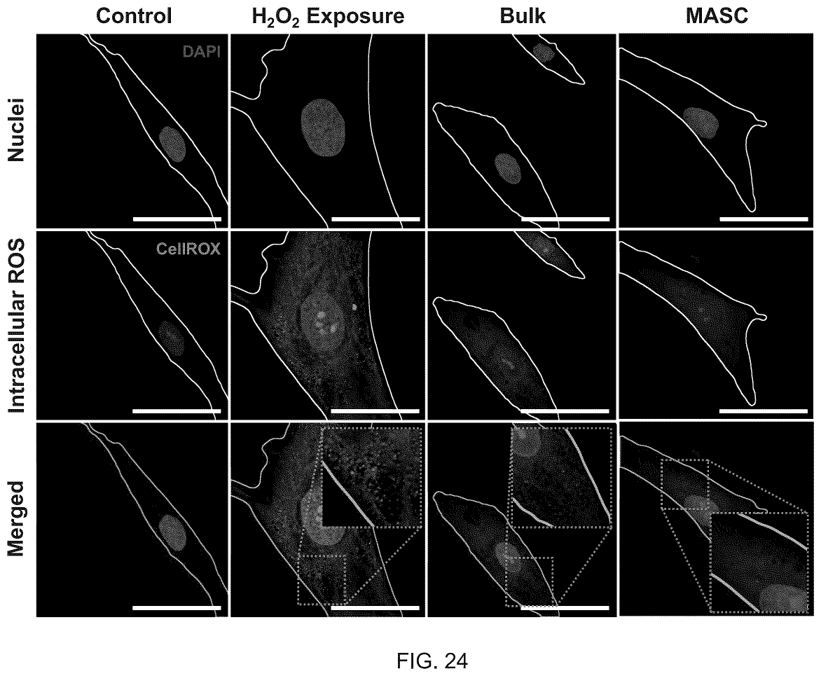

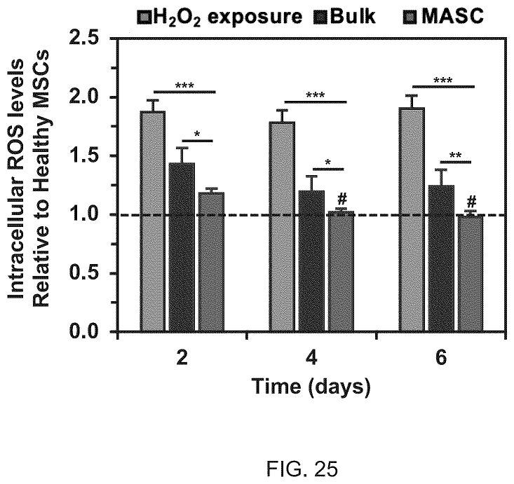

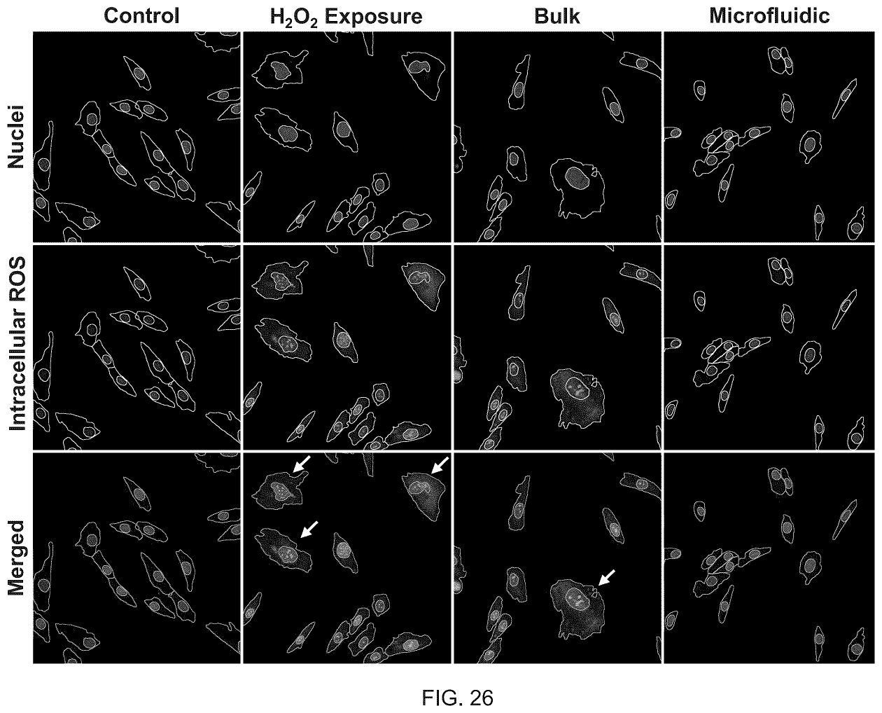

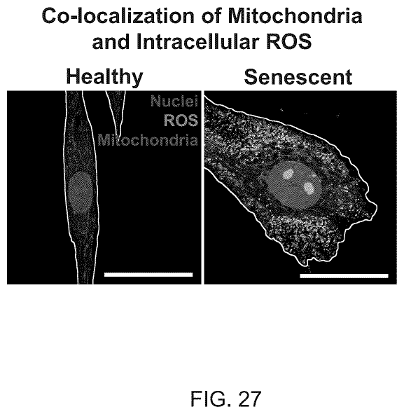

As discussed previously, minimal variation in antioxidant release can improve the therapeutic efficacy by maintaining cellular homeostasis. As such, it was tested whether MASC is more effective in reverting ROS-driven senescence in human adipose-derived MSCs and preserving cellular paracrine secretion activity compared to bulk NAC crystals. Senescent MSCs were created through H2O2 exposure for 5 days (FIG. 22). Then, MSCs were exposed to either antioxidant-free media (denoted as “H2O2 exposure”), bulk NAC crystals, or MASC for up to 6 days. To confirm that the H2O2 exposure condition did not induce cell death, a LIVE/DEAD assay was conducted (FIG. 9). No statistical difference in cell death between cells never exposed to H2O2 or exposed to 25 μm H2O2 daily for 5 days was discovered. In order to confirm that MASC is biocompatible, a range of MASC concentrations and the effect on MSC metabolic function was screened (FIG. 23). No statistical difference in metabolic function was observed at a concentration of 200 μm and lower when compared to MSCs never exposed to MASC. To illustrate the antioxidant activity of the crystals, the MSCs oxidative state was monitored through live-cell fluorescence staining of intracellular ROS levels (FIGS. 24-25; and FIG. 26). The mean intracellular ROS levels were over 1.8-times greater in the cell population exposed to H2O2 compared to normal cells. Furthermore, senescent cells showed increased green-fluorescent puncta, indicating intracellular oxidative stress levels in the mitochondria as marked by colocalization with a fluorescent mitochondrial stain (FIG. 27; and FIGS. 11-13). This observation is consistent with the literature characterization of senescent cells and matches the current hypothesis that ROS-mediated cellular senescence starts with mitochondrial dysfunction and ROS overproduction. When the cells were treated with either bulk NAC crystals or MASC, the mean intracellular green intensity decreased. While MASC restored the intracellular ROS levels to that of normal cells by day 4, cells treated with bulk crystals exhibited a 24% higher ROS level than normal cells.

### Characterization of MSC Senescence and Paracrine Signaling

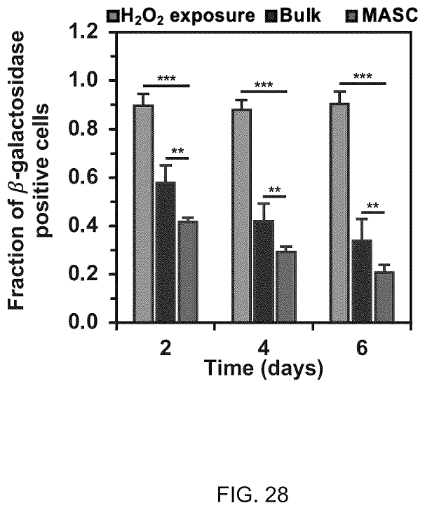

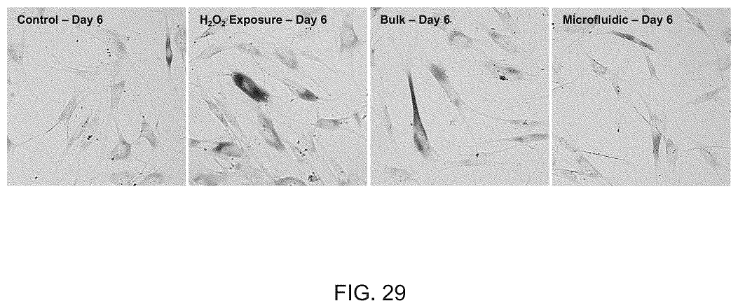

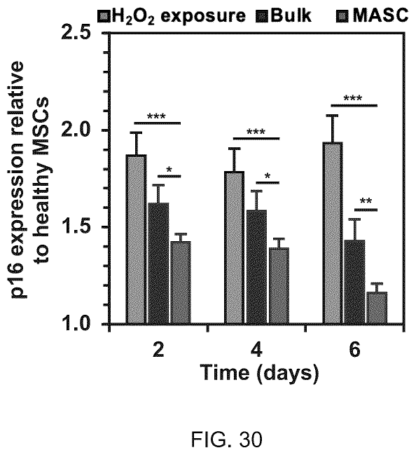

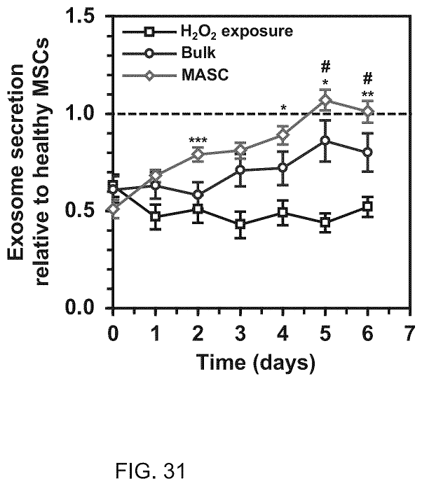

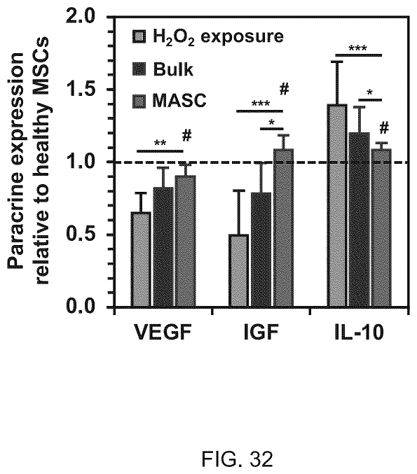

The degree of senescence was also characterized with two well established markers: 1) the fraction of β-galactosidase-positive cells (FIG. 28; and FIGS. 29) and 2) the gene expression level of tumor suppression protein, p16 (FIG. 30). Cells incubated with either NAC crystals exhibited a substantial decrease in the fraction of β-galactosidase positive cells and p16 gene expression. Interestingly, MASC greatly reduced the number of senescent cells compared to treatment with bulk crystals. After 6 days of treatment with MASC, the fraction of β-galactosidase-positive cells was reduced from 90% to 21%, and the p16 gene expression was reduced from 1.93 to 1.16, suggesting that control of the oxidative state can protect against cellular senescence. Treatment with bulk NAC crystals for 6 days only reduced the fraction of β-galactosidase-positive cells to 33.9% and the p16 gene expression to 1.30. The secretome of the MSCs was characterized to gauge their therapeutic potential by monitoring the exosome secretion levels (FIG. 31) and paracrine gene expression for vascular endothelial growth factor (VEGF), insulin-like growth MSCs (FIG. 32). MSCs exposed to H2O2 showed low exosome secretion, but NAC crystal treatment led to a significant recovery in exosome secretion following 6 days of treatment. In particular, cells treated with MASC restored exosome secretion levels equal to those never exposed to H2O2. A similar trend was observed for the gene expressions of VEGF, IGF, and IL-10. MASC treatment enabled cells to retain healthy baseline expression compared to NAC-free media and bulk NAC crystal treatment.

### Variation in MSCs Phenotypic Expression

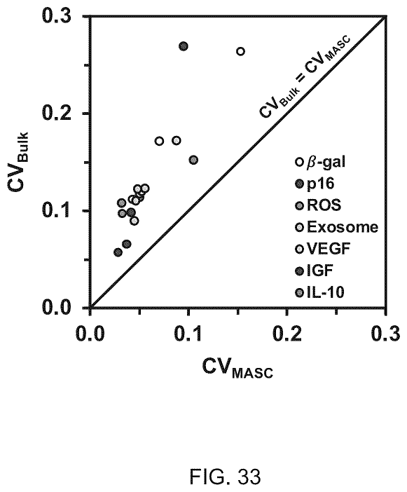

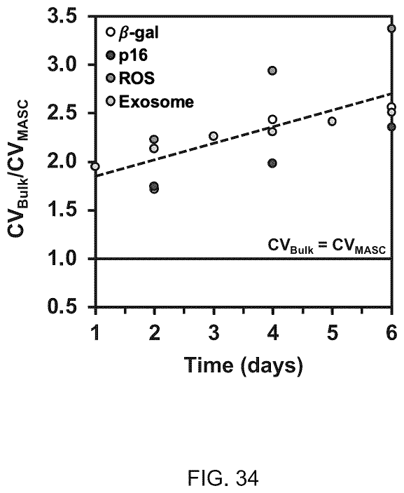

It was observed whether the CV in each experiment was smaller for MASC (CVMASC) than bulk NAC crystals (CVBulk). Plotting CVBulk/CVMASC for all in vitro experiments demonstrated two key trends (FIGS. 33-34): 1) a decrease in release variation leads to a decrease in the variation of cell state; and 2) CVBulk increases faster than CVMASC over time. This characterization helps to emphasize that drugs designed to control cellular homeostasis require predictable and uniform release but also have major implications in long-term treatment of an injury or disease. Heterogeneity in cell state negatively influences cellular homeostasis and can change the direction of disease pathogenesis.

### NAC Efficacy Based Only on Uniform Release

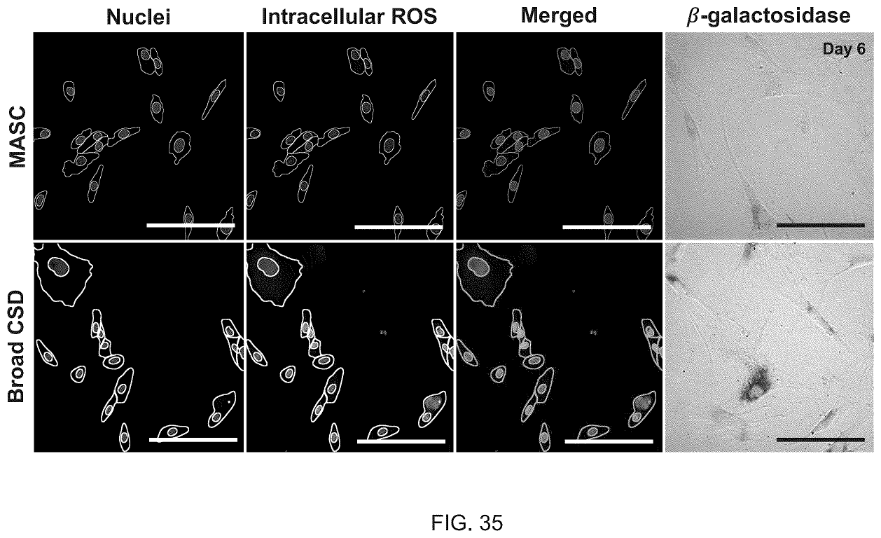

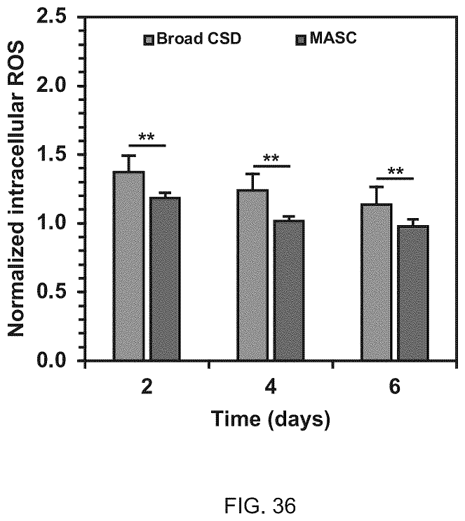

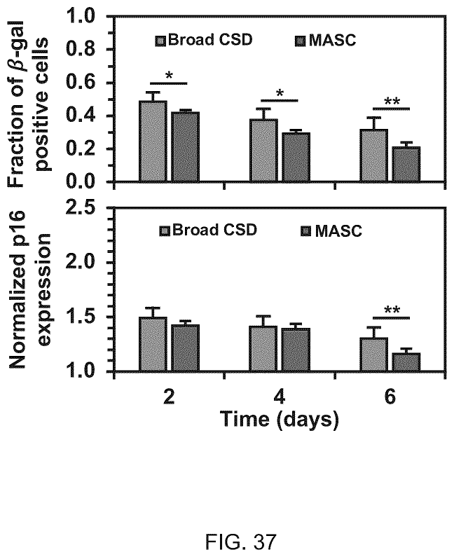

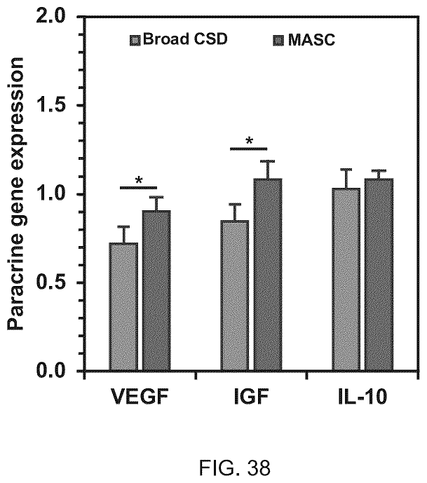

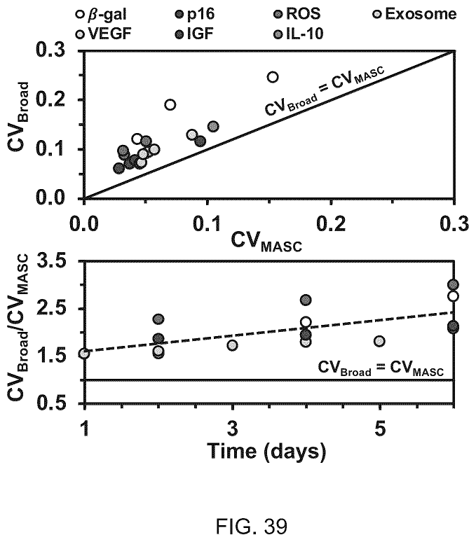

To confirm that the improved efficacy from MASC is linked to the CSD and release variation and not just the release rates, an NAC crystal (denoted as “broad CSD”) was engineered and repeated the MSC efficacy studies (FIGS. 5-7). The crystal assembly followed the same protocol previously described for fabricating bulk NAC crystals, except an additional crystal growth step was included. Following the 5 thermocycles and 12 h hold at 4° C., 200 μL of supersaturated NAC was added dropwise into the crystal suspension. This approach promotes crystal growth rather than the formation of new nuclei. The resulting broad CSD crystals show a similar release profile to MASC but have a larger size distribution and release variation than MASC. While the measured therapeutic differences between the broad CSD crystals and MASC are reduced, MASC still better preserves the healthy MSC state than the broad CSD crystals (FIGS. 35-39). Notably, the oxidative stress levels following MASC treatment are still lower and less variable, leading to cell function most similar to the healthy MSC population. Overall, translation of cell-derived secretome-based therapeutics into the clinical setting is heavily setback by functional cell heterogeneity. Cell heterogeneity can be caused by donor-to-donor variability as well as cell manufacturing approaches. The oxidative state of MSCs has major implications on phenotypic expression and needs to be controlled when manufacturing MSC-derived therapeutics. To date, most clinical protocols require extensive MSC expansion. Therefore, easy to implement and relatively cheap approaches to minimizing cell heterogencity are most actively pursued. Namely, “rejuvenating” media is used to preserve cellular homeostasis. Nonetheless, the antioxidant additives lose their activity prior to 48 h when media is conventionally changed. The MASC system of the present subject matter tackles this approach by extending antioxidant release. More interestingly, the MASC system minimizes burst release which can drive cells into antioxidative stress, lead to DNA damage, and promote cellular senescence. Suppressing variation in cell phenotypic expression allows for more predictable MSC secretion activities and, therefore, leads to reliable biologics manufacturing. Furthermore, this system can be directly applied to the manufacturing patient-derived therapeutics which add benefit of minimizing the probability of a host response.

### Example 5. Shear-Induced Nucleation of N-Acetylcysteine in Drops

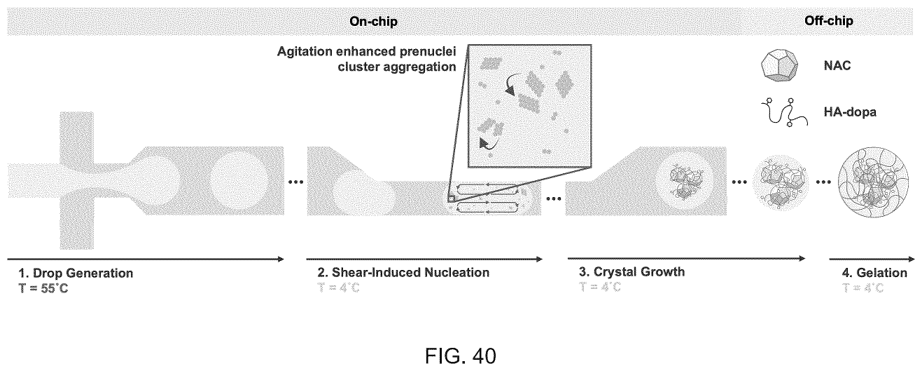

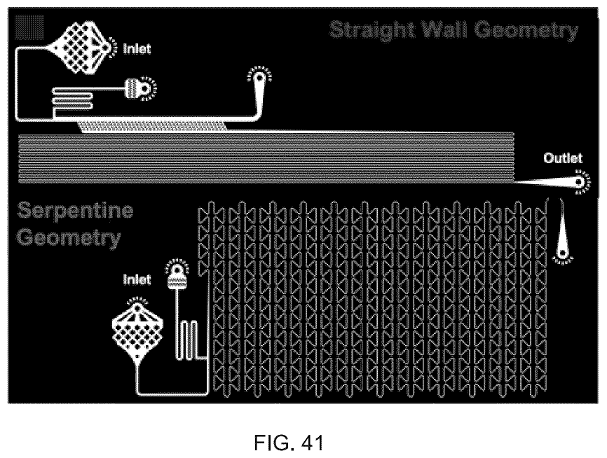

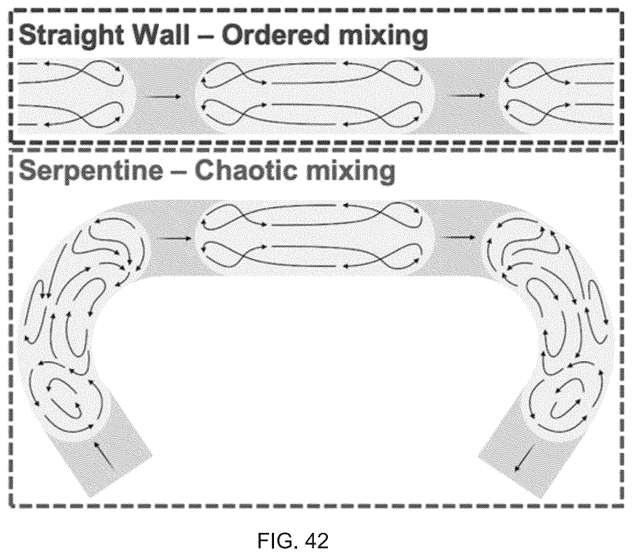

Droplet microfluidics was used to generate monodisperse hydrophilic drug crystals, ensuring a homogeneous antioxidant release profile. Specifically, NAC microdrops were prepared by emulsifying supersaturated NAC solution (364 mg/mL at 45° C.) mixed with 2.5 wt % hyaluronate-dopamine and 30 wt % PEGDA using a micro-drop generator (FIG. 40), followed by in-drop crystallization at 4° C. The inherent problem of slow crystallization, and low crystallization efficiencies of highly hydrophilic drugs in drops (˜ 20%) was addressed by devising a microfluidic channel design that introduces fluid agitation within the microdrops (FIGS. 41-42). It is believed that increased nucleation kinetics can result from prenuclei cluster collisions and the formation of larger clusters, which shorten the crystal induction time. Straight and serpentine channels were tested that have a smaller cross-section than microdrop size to identify the optimal device geometry for shear-induced crystallization (FIGS. 41-42). The resulting shear force at the interface of the drop and the narrowed channel walls can induce mixing in the drops, where the channel geometry can control the degree of mixing. In particular, the serpentine channel can cause chaotic mixing, while the straight channel makes an ordered orbital flow pattern. The device temperature was kept constant at 4° C. Such temperature decreases (from room temperature) act as the thermodynamic driving force for nucleation, where the shear-induced flow in drops kinetically influences nucleation probability in individual micro-drops.

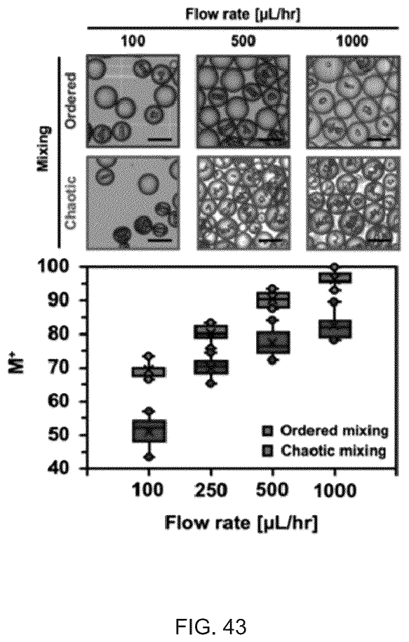

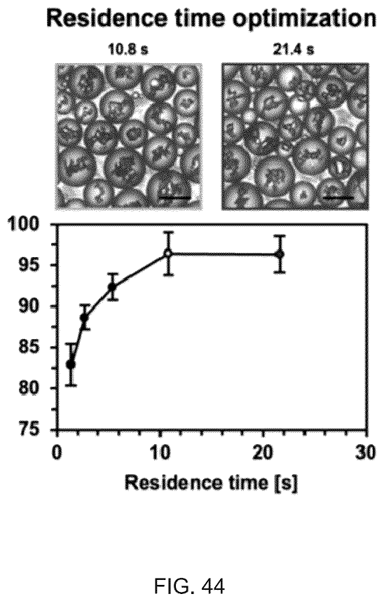

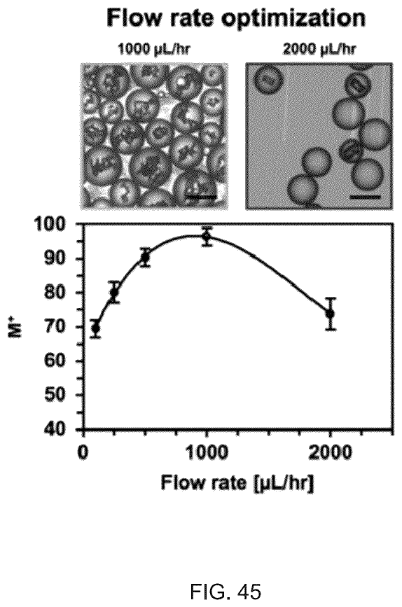

With the above configuration, the extent to which the microfluidic geometry of the shear-induced nucleation device and total flow rates regulate the fraction of drops containing at least one crystal (M⁺) was examined (FIG. 43). At the same flow rates, M⁺ was greater with the serpentine microchannel than with the straight wall channel. It is believed that the increased M⁺ is attributed to the chaotic mixing in the serpentine channel, which elevates the probability of prenuclei collision compared to the ordered mixing in the straight wall channel. As such, crystal-positive drops processed in the straight wall channels have fewer crystals than those processed in the serpentine channels. However, it is noted that increased shear in the serpentine channels may lead to nuclei fragmentation and result in a larger number of crystals in a single drop. With the serpentine channel, effects of on-chip residence time on M⁺ were screened. The total flow rate at 1,000 μL/hr was fixed, and the channel length was varied from 120 mm to 2,400 mm (FIG. 44). The increased channel length increased the residence time to 21.6 s. It was found that M⁺ reaches an equilibrium of 96.4 with a residence time of 10.8 s. At a fixed residence time of 10.8 s, the effects of the flow rate on M⁺ was examined because it was suspected that there is a critical shear rate that would serve as a barrier to nuclei formation. Increasing the flow rate from 100 μL/hr to 1,000 μL/hr resulted in the increase of M⁺ from 69.5 to 96.4 (FIG. 45). Further increase of the flow rate to 2,000 L/hr moderately decreased M⁺ to 73.8. In short, chip design and optimized flow conditions achieved close-to unity-crystallization efficiencies eliminating the need for removing drops without drug crystals, which would require sophisticated imaging systems and on chip-drop manipulation.

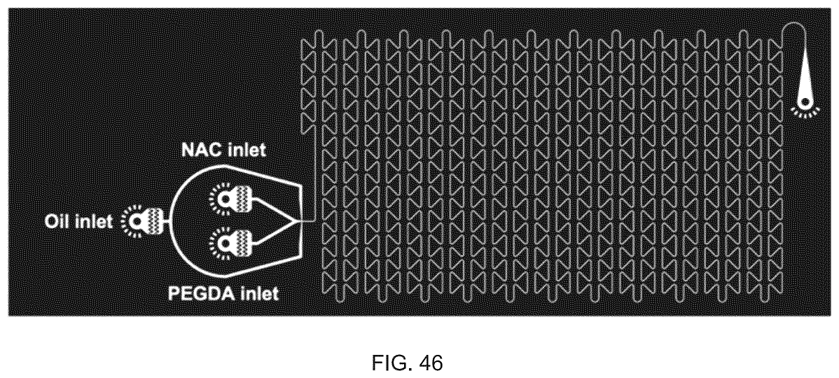

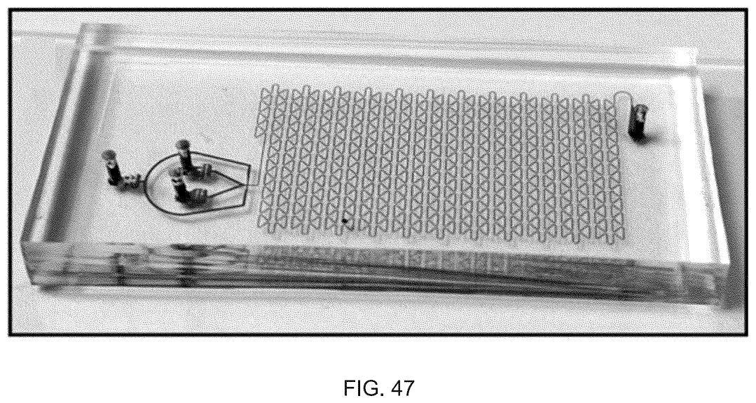

The final platform coupled microdrop generation with shear-induced nucleation, thereby streamlining droplet encapsulation of NAC drugs and drug crystallization in PEGDA microgels (μgels) (FIGS. 46-47). First, microdrops were made at the junction where the inlets meet, and then shear-induced mixing occurred in the serpentine geometry. This device avoids the drop reinjection step that can exacerbate drop merging while also making the in-drop crystallization process fully continuous. The fully continuous setup was able to yield crystal loaded drops of comparable quality.

### Example 6. In Situ Gelation of NAC Crystal-Loaded Microdrops and Release Analysis

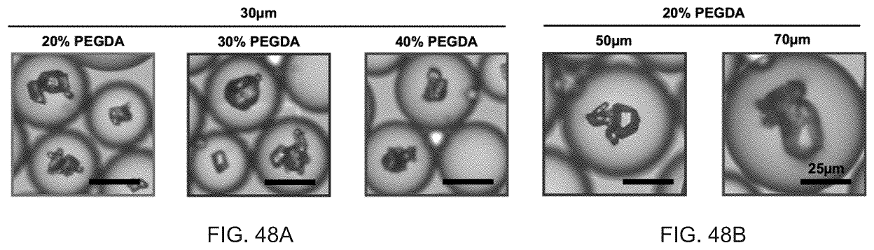

It is believed that the continual and uniform release of antioxidants will best promote ROS homeostasis. Therefore, rather than demulsifying the drops to harvest the NAC crystals, converting liquid drops to μgels adds a diffusion barrier and extends the release. The pre-gelled solution contained PEGDA (20-40 wt %) and 0.1% APS, while the continuous phase contained 0.4% TEMED. After drop generation and nucleation, drops were incubated at 4° C. to fully crystallize NAC for 6 hours. Finally, the gelation was initiated by heating the drops to 70° C. for 5 min and then the drops were cooled back to 4° C. to minimize crystal dissolution. After 12 hrs, the microdrops encapsulating NAC crystals were converted to μgels. The μgel structure was controlled, particularly permeability, by varying PEGDA concentration (FIG. 48a). Separately, the μgel diameter was altered from 30 to 50 and 70 μm by changing the geometry of the microdrop generator junction or the ratio of aqueous to oil flow rates.

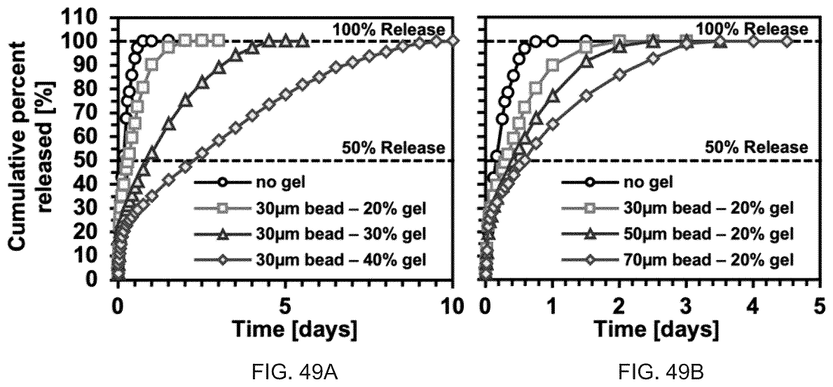

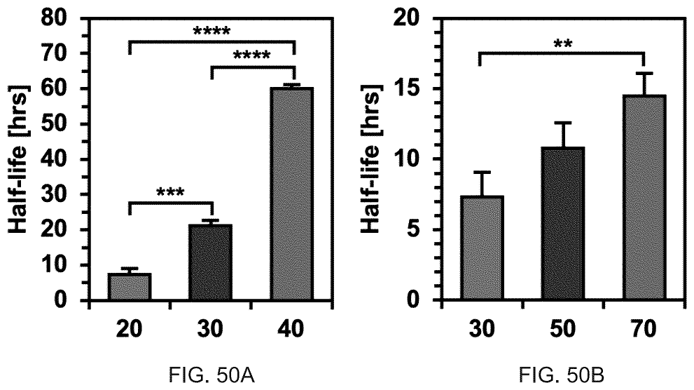

As expected, increasing PEGDA concentration significantly reduced the initial burst and extended the time for total NAC release proportional to the PEGDA concentration. (FIG. 49a). Accordingly, the time for half of the NAC to be released, denoted as half-life, was increased from 7 hrs to 60 hrs by increasing PEGDA concentration from 20 to 40% (FIG. 50a). The higher PEGDA wt % leads to an increase in the degree of crosslinking in the μgel and smaller pore size. As such, the diffusion constant for the gel drastically increased, resulting in prolonged release of NAC.

Separately, increasing the μgel diameter from 30 to 50 and 70 μm extended the time for μgels to release all NAC cargos as well as half-life (Figure s 49b, 50b). Increasing the starting drop size led to a slight increase in the NAC crystal size in the resulting gels. Therefore, larger NAC crystals can explain why increasing the μgel size would extend the NAC release time. Furthermore, since NAC dissolved from the crystal surface should diffuse into the bulk phase through tortuous nano-sized gel pores, this μgel may amplify the effect of crystal size on the dissolution rate. Based on these results, senescence spreading was controlled using the 30 μm-dimater μgel prepared by cross-linking 30 wt % PEGDA solution since NAC conditioning efficacy studies last for 6 days.

Other features, advantages and embodiments of the invention disclosed herein will be readily apparent to those exercising ordinary skill after reading the foregoing disclosure. In this regard, while specific embodiments of the invention have been described in considerable detail, variations and modifications of these embodiments can be effected without departing from the spirit and scope of the invention as described and claimed.

We claim:

1. A process for producing a crystalline antioxidant comprising:
providing an aqueous solution comprising:
a hydrophilic antioxidant; and
a polymer selected from hyaluronate-dopamine, alginate-dopamine, chitosan-dopamine, poly(ethylene glycol) dopamine and their derivatives, or mixtures thereof;
forming a liquid droplet of the aqueous solution, the droplet having a diameter, wherein polymer-induced nucleation of the antioxidant occurs in the liquid droplets;
crystallizing the crystalline antioxidant by performing at least one of:
(a) subjecting the liquid droplet to thermal cycling between a lower temperature and a higher temperature,
wherein antioxidant crystal growth occurs at the lower temperature and crystal dissolution occurs at the upper temperature; and
maintaining the thermal cycling until the droplet contains one or two antioxidant crystals, and
(b) subjecting the liquid droplet to shear deformation using a shear-induced mixing-on-a-chip device, wherein a probability of nucleation and crystallization of each droplet is increased.

2. The process of claim 1 wherein the droplet diameter is from 10 to 200 μm.

3. The process of claim 2 wherein the droplet diameter is from 10 to 25 μm.

4. The process of claim 1 wherein the hydrophilic antioxidant is selected from N-acetylcysteine, ascorbic acid, vitamin C, uric acid, and glutathione.

5. The process of claim 4 wherein the hydrophilic antioxidant is selected from N-acetylcysteine and ascorbic acid.

6. The process of claim 1 wherein the polymer is hyaluronate-dopamine.

7. The process of claim 1 wherein the lower temperature is from 4° C. to 25° C.

8. The process of claim 1 wherein the higher temperature is from 45° C. to 65° C.

9. The process of claim 1 wherein a temperature difference between the lower temperature and the higher temperature is from 20° C. to 60° C.

10. The process of claim 1 wherein the total number of cycles is 3 to 7.

11. The process of claim 1 further comprising a hold time of the cycle at the low temperature and a hold time of the cycle at the high temperature.

12. The process of claim 11 wherein the hold time of the cycle at the low temperature is from 20 to 60 min.

13. The process of claim 11 wherein the hold time of the cycle at the high temperature is from 10 to 30 min.

14. The process of claim 1 further comprising a total time for the temperature cycling.

15. The process of claim 14 wherein the total time is from 12 to 24 hrs.

16. The process of claim 1 wherein the concentration of the hydrophilic antioxidant in the aqueous solution is from 200 mg/mL to 800 mg/mL.

17. The process of claim 16 wherein the concentration of the hydrophilic antioxidant in the aqueous solution is from 300 mg/mL to 400 mg/mL.

18. The process of claim 1 wherein the concentration of the polymer in the aqueous solution is from 5 mg/mL to 80 mg/mL.

19. The process of claim 18 wherein the concentration of the polymer in the aqueous solution is from 15 mg/mL to 20 mg/mL.

20. The process of claim 1 wherein the thermal cycling is maintained until the droplet contains one crystal.

21. The process of claim 1 further comprising a hold time following the low temperature of the last cycle.

22. The process of claim 21 where the hold time is 10-14 hour.
